(19) United States
(12) Patent Application Publication
(10) Pub. No.: US 2025/0294115 A1
(43) Pub. Date: Sep. 18, 2025

(54) MULTI INPUT/OUTPUT SURGICAL SYSTEM WITH DISTRIBUTED AI PROCESSING

(71) Applicant: SoftAcuity Inc., Santa Barbara, CA (US)

(72) Inventors: Bruce Laurence Kennedy, Santa Barbara, CA (US); Johan Fornier, Kortrijk (BE); Matthew Wood, Santa Barbara, CA (US)

(21) Appl. No.: 19/080,740

(22) Filed: Mar. 14, 2025

Related U.S. Application Data
(60) Provisional application No. 63/566,154, filed on Mar. 15, 2024.

(51) Int. Cl. H04N 7/015; G06T 3/40; H04N 19/136; H04N 19/42

(52) U.S. Cl. CPC H04N 7/015; G06T 3/40; H04N 19/136; H04N 19/42

(57) ABSTRACT

A surgical video system having: at least one video source; at least one artificial intelligence platform, the at least one artificial intelligence platform having at least one artificial intelligence solution for processing video received from the at least one video source and providing at least one output; a switch coupling the at least one video source to the at least one artificial intelligence platform; and a display coupled to the switch and the artificial intelligence platform, the display further comprising a processor and at least one user input device. The display is configured to control the at least one artificial intelligence solution and to display video from the at least one video source along with output from the artificial intelligence platform.

### CROSS REFERENCE TO RELATED APPLICATION

This application claims priority from U.S. Provisional Patent Application No. 63/566,154, filed on Mar. 15, 2024, entitled MULTI INPUT/OUTPUT SURGICAL SYSTEM WITH DISTRIBUTED AI PROCESSING, the entire contents of which are hereby incorporated herein by reference.

### BACKGROUND

The present disclosure relates to devices used in surgery and, more particularly, to systems for using artificial intelligence (AI) to analyze surgical video.

Machine learning algorithms (also known as artificial intelligence or AI) have allowed for tremendous surgical improvements. Typically, surgical images can benefit from analysis by artificial intelligence, but the constraints of video processing and transmission limit the quality and quantity of video that can be subjected to artificial intelligence processing.

There is therefore a need for an improved system for processing ultra-high-definition, low latency surgical video using graphical processing units (GPU(s)) and artificial intelligence, enabling the flexible routing of video and/or sensor streams into multiple artificial intelligence solutions.

### SUMMARY

The present application is directed to a surgical video system comprising: at least one video source; at least one GPU-based artificial intelligence platform, the at least one artificial intelligence platform further comprising at least one artificial intelligence solution for processing video received from the at least one video source and providing at least one output; a switch coupling the at least one video source to the at least one artificial intelligence platform; and at least one display coupled to the switch and the artificial intelligence platform, the display further comprising a processor and at least one user input device. The at least one display is configured to control the at least one artificial intelligence solution and to display video from the at least one video source along with the output from the artificial intelligence platform.

The surgical video system may have a plurality of video sources. In an implementation, the display is further configured to control routing of video from at least one video source and video from the artificial intelligence platform. In an implementation, the artificial intelligence platform is configured to output an overlay for surgical video processed by the at least one artificial intelligence solution and the video from the at least one video source and any overlay generated by the artificial intelligence solution are combined in the at least one display.

The surgical video system may have a plurality of sensor data sources and video sources. The surgical video system may have a plurality of GPU-based artificial intelligence platforms performing distributed processing of at least one artificial intelligence solution. The video source may provide video at a resolution selected from the group consisting of 1080 p video, 4 K video and 8 K video and the system may be configured to transmit the video from the video source to the artificial intelligence platform and from the artificial intelligence platform to the at least one display in less than about 40 milliseconds. In an implementation, the system is configured to transmit the video from the video source to the artificial intelligence platform and from the artificial intelligence platform to the at least one display in less than about 20 milliseconds. In an implementation, the system is configured to transmit the video from the video source to the artificial intelligence platform and from the artificial intelligence platform to the at least one display in less than about 10 milliseconds.

The video may be transmitted to the artificial intelligence platform at a reduced resolution and the output from the artificial intelligence platform resized prior to display with the video from the at least one video source. The video may be transmitted to the artificial intelligence platform at a reduced frame rate and the output from the artificial intelligence platform multiplied prior to display with the video from the at least one video source. In an implementation the switch is configured to transmit video from the video source to the display without any output from the artificial intelligence platform if an error is detected from the artificial intelligence platform. The system may also have a plurality of encoders to encode video from the at least one video source and from the artificial intelligence platform; and a plurality of decoders to decode video from the switch; and the encoders and decoders may be configured to encrypt and decrypt the video for secure transmission.

The present application is also directed to a surgical video system comprising: at least one video source, the at least one video source producing video comprising a resolution of at least one of the group consisting of 1080 p video, 4 K video and 8 K video; a first video encoder coupled to the at one video source; at least one artificial intelligence platform, the at least one artificial intelligence platform further comprising at least one artificial intelligence solution for processing video received from the at least one video source and providing at least one output; a second video encoder coupled to at least one artificial intelligence platform; a switch coupling the at least one video source to the at least one artificial intelligence platform; at least one video decoder coupled to the switch; and at least one display coupled to the switch and the artificial intelligence platform, the display further comprising a processor and at least one user input device. The at least one display is configured to control the at least one artificial intelligence solution and to display video from the at least one video source along with output from the artificial intelligence platform.

In an implementation, the artificial intelligence platform is configured to output an overlay for surgical video processed by the at least one artificial intelligence solution and the video from the at least one video source and any overlay generated by the artificial intelligence solution are combined in the at least one display. The surgical video system may also have a plurality of sensor data sources and video sources. The surgical video system may be configured to transmit the video from the video source to the artificial intelligence platform and from the artificial intelligence platform to the at least one display in less than about 20 milliseconds. The surgical video system may be configured to transmit the video from the video source to the artificial intelligence platform and from the artificial intelligence platform to the at least one display in less than about 10 milliseconds.

In an implementation, the switch is configured to transmit video from the video source to the display without any output from the artificial intelligence platform if an error is detected from the artificial intelligence platform. In an implementation, the encoders and decoders encrypt and decrypt the video for secure transmission.

These and other features are described below.

### BRIEF DESCRIPTION OF THE DRAWINGS

The features, aspects and advantages of the present invention will become better understood with regard to the following description, appended claims and accompanying figures wherein:

FIG. 1 is a schematic diagram of a surgical system for enabling the use of artificial intelligence in surgery according to an implementation;

FIG. 2 is a schematic diagram of a surgical system for enabling the use of artificial intelligence in surgery according to an additional implementation; and

FIG. 3 is a screenshot of an interface for controlling an artificial intelligence feature according to an implementation.

### DETAILED DESCRIPTION

In the following description of the preferred implementations, reference is made to the accompanying drawings which show by way of illustration specific implementations in which the invention may be practiced. Wherever possible, the same reference numbers will be used throughout the drawings to refer to the same or like parts. It is to be understood that other implementations may be utilized, and structural and functional changes may be made without departing from the scope of this disclosure.

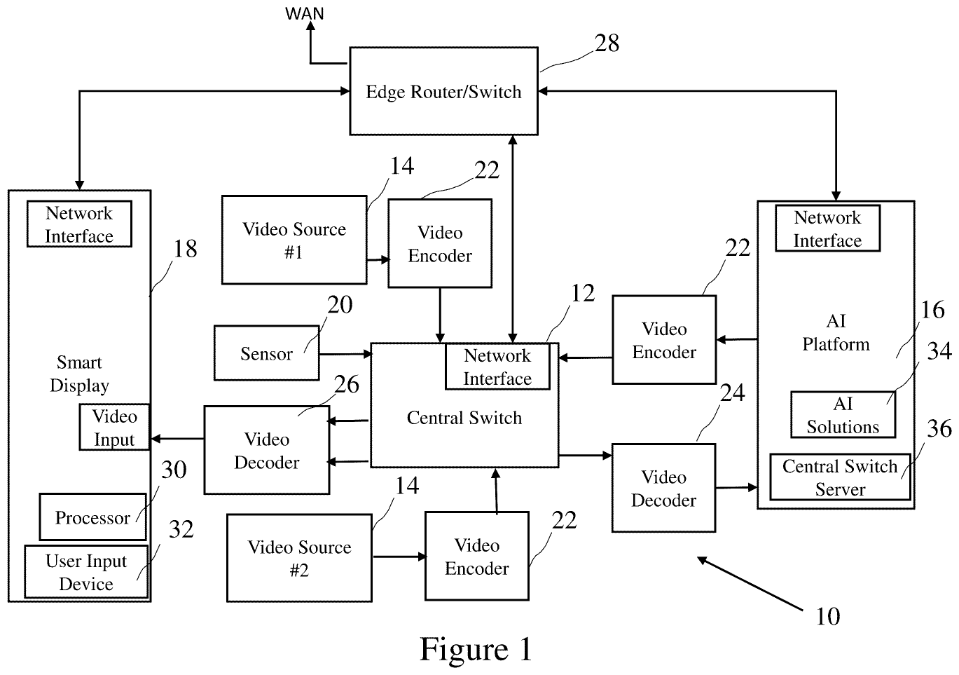

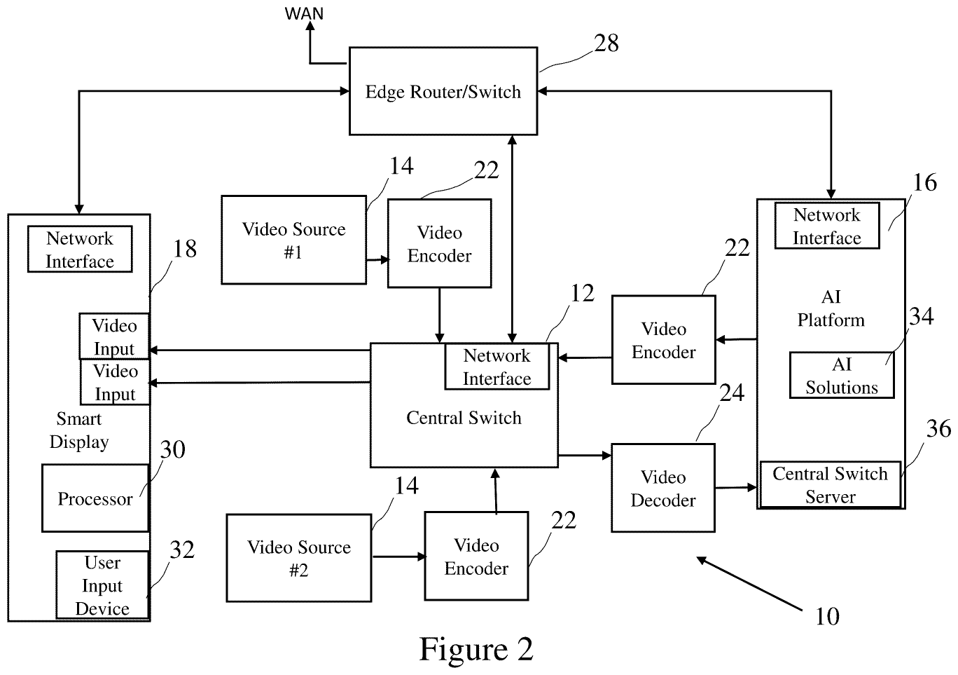

As shown in FIGS. 1 and 2, according to an implementation, a system 10 for artificial intelligence processing of ultra-high-definition surgical video has a central high throughput switch 12, such as an optical fiber switch, for routing ultra-high-definition surgical video from video sources 14 to a GPU-based artificial intelligence platform 16 for processing and from the artificial intelligence platform to a smart display 18. The ultra-high-definition (UHD) surgical video may be, for example, 1080 p video, 4 K video and 8 K video. The UHD surgical video may have a frame rate of 30 fps and more preferably 60 fps.

The video sources 14 may be, for example, endoscopic video cameras or operating room cameras. Additionally, there may be other sensor data streams 20, for example and without limitation audio data or telemetry streams from connected surgical equipment. The artificial intelligence platform 16 may be an IGX Platform by Nvidia, 2788 San Tomas Expressway, Santa Clara, CA 95051. In an implementation, the IGX Platform comprises a discreet GPU (dGPU) with a Holoscan Stack running Nvidia AI Enterprise Software.

In an implementation, ultra-high-definition video from the video sources 14 is encoded by a video encoder 22 prior to being transmitted to the central switch 12. The encoded video is decoded by a video decoder 24 prior to being transmitted to the artificial intelligence platform 16. In an implementation, following artificial intelligence inference processing, the ultra-high-definition video is encoded by a video encoder 22 prior to being transmitted to the central switch 12. The processed ultra-high-definition video is then decoded by a video decoder 26, effectively combining an artificial intelligence inference output overlayed with the low-latency source ultra-high-definition video, prior to transmission to the smart display 18. In an additional implementation, as shown in FIG. 2, the smart display 18 is configured to decode and composite to combine the artificial intelligence platform output overlay with the lower-latency source ultra-high-definition video. In an additional implementation, the central switch 12 is controlled by a central switch server 36 in the AI Platform 16 to combine the artificial intelligence platform output overlay with the low-latency source ultra-high-definition video prior to transmission to the smart display 18.

In an implementation, the central switch 12 is a managed fiber switch. For example, the central switch 12 may be an optical fiber switch made by Cisco Systems, Inc., 300 East Tasman Drive, San Jose, CA 95134. The central switch 12 may be connected to the encoders 22 and decoders 24 by multiple connections. In an implementation, the central switch 12 is connected to the encoders 22 and decoders 24 by two 10 Gigabit fiber lines. In an implementation, the video encoders and decoders are made by Barco, Inc., 3059 Premiere Parkway Suite 400, Duluth, Georgia 30097.

The system 10 is configured to transmit surgical video to the artificial intelligence platform 16 and from the artificial intelligence platform to the smart display 18 at fast enough speeds to prevent lag to a user. In an implementation, the system 10 is configured to transmit ultra-high-definition video from the video sources 14 to the artificial intelligence platform 16 and from the artificial intelligence platform in less than about 40 milliseconds, and more preferably less than about 20 milliseconds, and more preferably less than about 10 milliseconds.

In an implementation, to speed up processing by the artificial intelligence platform, the video from the central switch 12 is transmitted to the artificial intelligence platform at a reduced resolution. In an implementation, to speed up processing by the artificial intelligence platform, the video from the central switch 12 is transmitted to the artificial intelligence platform at a reduced frame rate. In an implementation, the artificial intelligence platform output overlay is returned at a lower resolution than the native ultra-high-definition video from the video sources 14 and the artificial intelligence platform output overlay is rescaled when combined with the native ultra-high-definition video from the video sources 14. In an implementation, the artificial intelligence platform output overlay is returned at a lower frame rate than the native ultra-high-definition video from the video sources 14 and the artificial intelligence platform output overlay frame rate is multiplied when combined with the native ultra-high-definition video from the video sources 14.

In an implementation, the smart display 18 is also connected to a router 28 that is connected to the central switch 12 and the artificial intelligence platform 16. The connections between the smart display 18, router 28, central switch 12 and the artificial intelligence platform 16 may be, for example, ethernet connections. The router 28 may be connected to the Internet and may be used to download software updates for the smart display 18, central switch 12 and the artificial intelligence platform 16.

In an implementation, the smart display 18 has a processor 30 and at least one user input device 32, such as a touch screen. In an implementation, the processor 30 is coupled to an audio subsystem. The audio subsystem has at least one microphone and at least one speaker. The audio subsystem may contain an array of microphones. In an implementation, the audio subsystem contains two microphones configured for stereo. In an implementation, the audio subsystem contains five microphones. The microphones may be positioned at various points along an outer edge of the smart display 18. The microphones may be configured to beam steer and cross noise cancel.

In an implementation, the processor 30 is coupled to a visualization subsystem containing at least one camera, such as for capturing images of an operating room or users of the smart display 18. The visualization subsystem may have multiple cameras. In an implementation, the visualization subsystem has stereo cameras for assisting with room situational awareness or facial recognition. In an implementation, at least one camera is a time-of-flight camera. In an implementation, the visualization subsystem has two cameras for stereo imaging. In an additional implementation, the visualization subsystem has two cameras for three-dimensional image and video capture.

The smart display 18 functions as a control interface for artificial intelligence solutions 34 running on the artificial intelligence platform 16. The smart display 18 is coupled to the artificial intelligence platform 16 through the router 28. The smart display 18 utilizes software on the artificial intelligence platform 16 to route video from video sources 14 to sinks (such as displays and storage devices), select artificial intelligence solutions 34, and for controlling settings associated with the artificial intelligence solutions (such as, for example, color and transparency). Artificial intelligence solutions 34 that may be used include, for example and without limitation, a surgical tool detection solution, a colonoscopy polyp detection tool, anatomical anomaly detection tools, phase of surgery detection tools, retrieval augmented generation for instructions for use (IFU) queries, and others.

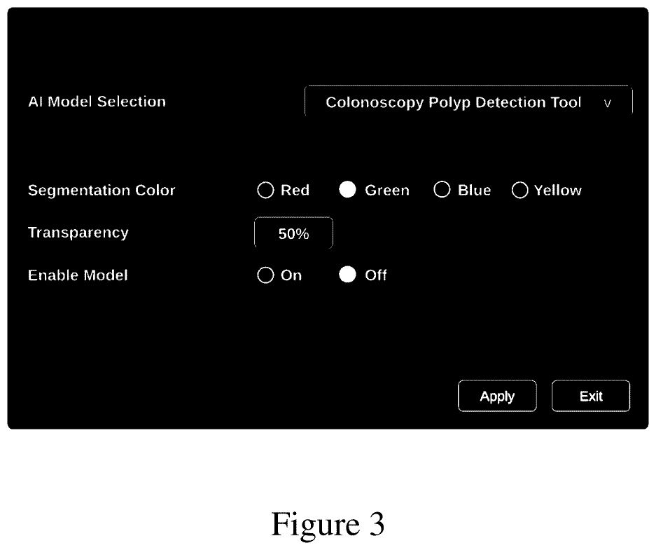

As shown in FIG. 3, in an implementation, a graphical user interface is presented to a user on the smart display 18, with drop down boxes for selecting certain artificial intelligence solutions to be performed on the surgical video. Additionally, radio buttons, checkboxes, or further drop down menus may be presented to a user for configuring the artificial intelligence solutions being performed on the surgical video. Additionally, the smart display 18 may allow for control of the artificial intelligence solutions 34 running on the artificial intelligence platform 16 using voice control. Additionally, the smart display 18 functions to control the routing of the video streams from the video sources 14 to the artificial intelligence platform 16 and where the output from the artificial intelligence platform is routed, such as to a display or a storage device.

The system 10 is scalable in that additional video sources 14 and additional sensor sources 20 and additional displays may be added to the system. The smart display 18 is usable to route video or data streams from the additional video or sensor sources to the artificial intelligence platform 16 and to the additional displays. The system is also scalable in that additional artificial intelligence nodes may be added for distributed processing, additional artificial intelligence solutions or to service more video sources 14 and additional displays. Additionally, the artificial intelligence platform 16 may be connected to the internet directory or indirectly, such as through the router 28 to allow for updating of existing artificial intelligence solutions 34 or the downloading of additional artificial intelligence solutions. Outputs from the artificial intelligence platform 16 may be captured and stored, such as for reference or learning.

In implementation, the system 10 keeps native video from the video source 14 separate from any overlay generated by an artificial intelligence solution 34. The native video from the video source 14 and any overlay generated by the artificial intelligence solution 34 are combined in the video decoder 26, in the smart display 18, or in the central switch 12. In an implementation, the processor 30 (or a field programmable gate array) of the smart display 18 combines the native video from the video source 14 and any overlay generated by the artificial intelligence solution 34.

By keeping the native video from the video source 14 and any overlay generated by the artificial intelligence solution 34 separate, any video latency is not affected by the artificial intelligence processing and the surgical video is protected from hardware crashes, software crashes or artificial intelligence solution glitches. In an implementation, if there is an error with the artificial intelligence platform 16 or the artificial intelligence solution 34, the system 10 is configured to transmit the low latency ultra-high-definition video from the video sources 14 to the smart display 18 to ensure that timely surgical video is available to a viewer. In an implementation, the smart display 18 is configured to display video from multiple video sources 14 simultaneously.

The system 10 may be linked to hospital networks, databases, and outside data processing. The system 10 may be linked to a picture archiving and communication system (PACS) either inside or outside of a hospital for obtaining or storing patient information. Additionally, the system may be linked to an electronic medical record (EMR)/electronic health record (HER) server either inside or outside of a hospital for obtaining or storing patient information. Additionally, system 10 may be linked to a cloud-based storage system.

Advantageously, the system 10 distributes artificial intelligence processing to remote artificial intelligence platforms 16 and artificial intelligence solutions 34 while ensuring distribution of ultra-low-latency transmission of video from the video sources 14 to the display 18. In an implementation, the system uses end-to-end encryption, to ensure data integrity, security, and privacy when routing sensitive surgical content. For example, the encoders 22 and decoders 24, 26 may be used to encrypt and decrypt video from the video sources 14.

There is disclosed in the above description and the drawings, a surgical video system that fully and effectively overcomes the disadvantages associated with the prior art. However, it will be apparent that variations and modifications of the disclosed implementations may be made without departing from the principles of the invention. The presentation of the implementations herein is offered by way of example only and not limitation, with a true scope and spirit of the invention being indicated by the following claims.

Any element in a claim that does not explicitly state “means” for performing a specified function or “step” for performing a specified function, should not be interpreted as a “means” or “step” clause as specified in 35 U.S.C. § 112.

What is claimed is:

1. A surgical video system comprising:
at least one video source;
at least one artificial intelligence platform, the at least one artificial intelligence platform further comprising at least one artificial intelligence solution for processing video received from the at least one video source and providing at least one output;
a switch coupling the at least one video source to the at least one artificial intelligence platform; and
at least one display coupled to the switch and the artificial intelligence platform, the display further comprising a processor and at least one user input device;
wherein the at least one display is configured to control the at least one artificial intelligence solution and to display video from the at least one video source along with output from the artificial intelligence platform.

2. The surgical video system of claim 1 further comprising a plurality of video sources.

3. The surgical video system of claim 1 wherein the display is further configured to control routing of video from the at least one video source and output from the artificial intelligence platform.

4. The surgical video system of claim 1 wherein the artificial intelligence platform is configured to output an overlay for surgical video processed by the at least one artificial intelligence solution and wherein the video from the at least one video source and any overlay generated by the artificial intelligence solution are combined in the at least one display.

5. The surgical video system of claim 1 further comprising a plurality of sensor data sources and video sources.

6. The surgical video system of claim 1 further comprising a plurality of GPU-based artificial intelligence platforms performing distributed processing of at least one artificial intelligence solution.

7. The surgical video system of claim 1 wherein the video source provides video at a resolution selected from the group consisting of 1080 p video, 4 K video and 8 K video and wherein the system is configured to transmit the video from the video source to the artificial intelligence platform and from the artificial intelligence platform to the at least one display in less than about 40 milliseconds.

8. The surgical video system of claim 7 wherein the system is configured to transmit the video from the video source to the artificial intelligence platform and from the artificial intelligence platform to the at least one display in less than about 20 milliseconds.

9. The surgical video system of claim 7 wherein the system is configured to transmit the video from the video source to the artificial intelligence platform and from the artificial intelligence platform to the at least one display in less than about 10 milliseconds.

10. The surgical video system of claim 1 wherein video is transmitted to the artificial intelligence platform at a reduced resolution and the output from the artificial intelligence platform is resized prior to display with the video from the at least one video source.

11. The surgical video system of claim 1 wherein video is transmitted to the artificial intelligence platform at a reduced frame rate and the output from the artificial intelligence platform is multiplied prior to display with the video from the at least one video source.

12. The surgical video system of claim 1 wherein the switch is configured to transmit video from the video source to the display without any output from the artificial intelligence platform if an error is detected from the artificial intelligence platform.

13. The surgical video system of claim 1 further comprising: a plurality of encoders to encode video from the at least one video source and from the artificial intelligence platform; and a plurality of decoders to decode video from the switch; and wherein the encoders and decoders encrypt and decrypt the video for secure transmission.

14. A surgical video system comprising:
at least one video source, the at least one video source producing video comprising a resolution of at least one of the group consisting of 1080 p video, 4 K video and 8 K video;
a first video encoder coupled to the at one video source;
at least one artificial intelligence platform, the at least one artificial intelligence platform further comprising at least one artificial intelligence solution for processing video received from the at least one video source and providing at least one output;
a second video encoder coupled to at least one artificial intelligence platform;
a switch coupling the at least one video source to the at least one artificial intelligence platform;
at least one video decoder coupled to the switch; and
at least one display coupled to the switch and the artificial intelligence platform, the display further comprising a processor and at least one user input device;
wherein the at least one display is configured to control the at least one artificial intelligence solution and to display video from the at least one video source along with output from the artificial intelligence platform.

15. The surgical video system of claim 14 wherein the artificial intelligence platform is configured to output an overlay for surgical video processed by the at least one artificial intelligence solution and wherein the video from the at least one video source and any overlay generated by the artificial intelligence solution are combined in the at least one display.

16. The surgical video system of claim 14 further comprising a plurality of sensor data sources and video sources.

17. The surgical video system of claim 14 wherein the system is configured to transmit the video from the video source to the artificial intelligence platform and from the artificial intelligence platform to the at least one display in less than about 20 milliseconds.

18. The surgical video system of claim 14 wherein the system is configured to transmit the video from the video source to the artificial intelligence platform and from the artificial intelligence platform to the at least one display in less than about 10 milliseconds.

19. The surgical video system of claim 14 wherein the switch is configured to transmit video from the video source to the display without any output from the artificial intelligence platform if an error is detected from the artificial intelligence platform.

20. The surgical video system of claim 14 wherein the encoders and decoders encrypt and decrypt the video for secure transmission.
